(19) United States
(12) Patent Application Publication
(10) Pub. No.: US 2024/0327310 A1
(43) Pub. Date: Oct. 3, 2024

(54) INSECT LARVA TRANSPORT CONTAINER AND BIOREACTOR FOR TREATING ORGANIC WASTE

(71) Applicant: Bybug SpA, Coquimbo (CL)

(72) Inventors: DANIEL TRONCOSO ESPINOSA, Coquimbo (CL); JOSÉ IGNACIO DEL SOLAR BOU, Coquimbo (CL); GERARDO BLUSKE MOSCOSO, Coquimbo (CL); ROCIO DEL CARMEN ESPINOSA TORRES, Coquimbo (CL); RICARDO FELIPE GUTIERREZ GARCIA, Coquimbo (CL); VALENTINA MUÑOZ ZAPATA, Coquimbo (CL); FRANCISCO JARA MONTECINOS, Coquimbo (CL); MACARENA URIARTE OSSANDÓN, Coquimbo (CL); FRANCISCO JAVIER ALTIMIRAS GONZALEZ, Coquimbo (CL); MATÍAS GERMAN BUSCHMANN ESPINOZA, Coquimbo (CL); MARTHA ADRIANA HAASE MALIG, Coquimbo (CL)

(73) Assignee: Bybug SpA, Coquimbo (CL)

(21) Appl. No.: 18/625,224

(22) Filed: Apr. 3, 2024

(30) Foreign Application Priority Data
Apr. 3, 2023 (CL) 202300966

(51) Int. Cl. C05F 3/06; A01K 67/033; A01M 1/02; A01M 1/10; B09B 3/35; B09B 3/50

(52) U.S. Cl. CPC C05F 3/06; A01K 67/033; A01M 1/02; A01M 1/10; B09B 3/35; B09B 3/50

(57) ABSTRACT

The present invention relates to the technical field of processing organic waste to convert it to useful products, such as compost or fertilizers and proteins obtained from insect larvae that are used to convert organic waste to animal feed and food for human beings. Specifically, the present invention relates to an egg laying prompting reservoir, egg germinator and larva transporter, and a bioreactor that converts organic waste to high value-added goods using the insect larvae from the transporter.

### CROSS-REFERENCE TO RELATED APPLICATIONS

This application is a US Non-Provisional application which claims priority from Chile Application No. 202300966 filed on Apr. 3, 2023, the entire contents of which are incorporated by reference.

### TECHNICAL FIELD

The present invention relates to the technical field of processing organic waste to convert it to useful products, such as compost or fertilizers and proteins obtained from insect larvae that are used to convert organic waste to animal feed and food for human beings. Specifically, the present invention relates to an egg laying prompting reservoir, egg germinator and larva transporter, and a bioreactor that converts organic waste to high value-added goods using the insect larvae from the transporter.

### BACKGROUND OF THE INVENTION

Organic waste is waste that is generated in the course of human activities and industrial processes, for example, in the agricultural, livestock or any other similar kind of industry, also including organic waste from household waste garbage.

This waste must be carefully handled to be moved to landfills where it decomposes without yielding any benefits. This degradation may take several hundred years and, during its decomposition, emits hundreds of tons of methane to the atmosphere, becoming, therefore, a contaminant.

However, different processes to treat this waste and convert it to a material with economic value supplementing the income of the industry that generated the waste have been recently developed.

Thus, for example, WO2022269563A1 discloses A method for treating waste water containing biodegradable organic substances by feeding the liquid to be treated to a process reactor containing black soldier fly larvae to metabolize the organic substances contained in the liquid to be treated, characterized by the insertion into the process reactor of a volumetric support bed made of solid non-biodegradable material, designed to support the larvae by allowing them on the one hand to come into contact with the liquid to be treated and on the other to move based on their metabolic needs, plunging in the liquid to feed themselves and re-emerging to breathe.

WO22093073 discloses a method for recycling raw plant material into feed for agricultural animals, aquaculture facilities and fertilizer production. Eggs of Hermetia illucens flies are incubated on a nutrient medium until the emergence of larvae. The larvae are used to colonize an entirely untreated ground substrate of softwood with a density of 10 to 100 specimens/cm² (the effective density of colonization depends on the size of the larvae). The larvae perform direct bioconversion of the substrate within 3-7 days at a temperature of 25 to 29° C. The substrate is placed into stackable reservoirs, the biomass of the larvae is separated from the resultant biocompost, and heat treatment and packaging are performed. The method makes it possible, without additional pretreatment of hard-to-decompose softwood waste, to obtain biocompost and a high-quality product in the form of insect biomass with a high (more than 40%) protein content, enriched with trace elements and organic substances, for direct use as fertilizer.

WO22053726 discloses a method for convert organic waste and by-products to valuable products and raw materials as fertilizers and insect larvae with high levels of proteins, lipids and wide amino acids. To that effect, the process starts with receiving and homogenizing the waste to be distributed together with larval biomass in metabolizing trains such that the larval biomass metabolizes the waste converting them to compost, whereas the adult larvae have a high protein content allowing their use such that they are first subjected to a drying process before being separated through screening and sieving.

In general, known in the prior art are processes such as the one shown in FIG. 1, where organic waste (1), together with insect larvae (2) are fed to a bioreactor (3) to obtain a compost or agricultural fertilizer (4) and protein (5) to produce animal feed.

To carry out these processes, the present invention proposes providing a larva and insect transport container and, in addition, providing a bioreactor to generate compost or (a) fertilizer and protein material for feeding animals.

### BRIEF DESCRIPTION OF THE DRAWINGS

The drawings below are included for the invention to be better understood, and they are an integral part hereof and show one of the preferred embodiments.

FIG. 1 is a block diagram showing the input and output products in the bioreactor of the present invention.

FIG. 2 is an exploded view of the transport container of the present invention.

FIG. 3 is a perspective view of the fabric tightening member used in the carrier container of the present invention.

FIG. 4 shows a side view of the fabric tightening member used in the carrier container of the present invention.

FIG. 5 is a perspective view of the rectangular insect egg laying trap used in the transport container of the present invention.

FIG. 6 shows is a perspective view of the egg and larva collecting tray used in the transport container of the present invention.

FIG. 7 shows a detail of the rectangular pillar of the tray used in the transport container of the present invention.

FIG. 8 shows is a perspective view of the lid used in the transport container of the present invention.

FIG. 9 is a front view of the transport container of the present invention.

FIG. 10 is a side view of the transport container of the present invention.

FIG. 11 is a top plan view of the transport container of the present invention.

FIGS. 12 to 15 show an assembly sequence of the transport container of the present invention.

FIG. 16 is a perspective view of the bioreactor of the present invention.

FIG. 17 is a right side view of the bioreactor of the present invention.

FIG. 18 is a right-side view without the perimeter walls of the bioreactor of the present invention.

FIG. 19 is a front view of the bioreactor of the present invention.

FIG. 20 is a front view without the perimeter walls of the bioreactor of the present invention.

FIG. 21 is a left-side view of the bioreactor of the present invention.

FIG. 22 is a left-side view without the perimeter walls of the bioreactor of the present invention.

FIG. 23 is a rear view of the bioreactor of the present invention.

FIG. 24 is a rear view without the perimeter walls of the bioreactor of the present invention.

FIG. 25 is a top plan view of the bioreactor of the present invention.

FIG. 26 shows a perspective view of the transport container 6 in its fully assembled state.

FIG. 27 shows a top down view of the rectangular trap with the plurality of slits that have eggs in them.

### DETAILED DESCRIPTION

The present invention relates to an insect egg and larva transport container allowing egg laying, larva germination and reception to be transported, together with a nutrient substrate to a bioreactor. Said bioreactor has an organic waste processing unit wherein said waste is ground, homogenized and texturized and then transferred to a tray where the insect larvae are placed, allowing said larvae to act and convert organic waste to compost or a fertilizer.

### Insect Larva Transport Container

The present invention is comprised, firstly, of a transport container (6) for insect larvae, which is made up of a box (7) having an inner floor (8) for piling placing the organic waste, which acts as an insect bait.

The transport container (6) has a lid (9) made up of a lid frame (9c) which has a central discharge (11). In the corners of the lid frame (9c), vertical pillars (10) emerge that have a concave cavity (10a) towards the interior area to house a lower portion (7a) of the box (7) to facilitate the stacking of the transport containers. (6. The inner corner of the lid frame (9c) is contoured by a perimeter flange (9a) having at least one pair of rectangular recesses (9b) to accommodate an upper projection (13a) of at least one pair of rectangular pillars (13) located at the outer corners of an egg and larva collecting tray (12). The rectangular recesses (9b) have an inclined inner face that connects with an inclined lower face (13e) of said rectangular pillars (13), thus enabling the collecting tray (12) to hang from the lid (9). See FIG. 13 wherein the collecting tray is shown hanging at a level that is below the level of the lid.

As shown in FIG. 7, the inner face (13b) of each rectangular pillar (13) has a vertical rib (13c) at the center for said pillars to be rigid. At mid height of the inner face (13b) of each vertical pillar (13) is a horizontal plate (13d) that serves as a support for a rectangular trap (14) having a plurality of slits (15) intended to catch the eggs and larvae that then fall into the collecting tray (12). The upper face of the rectangular trap has a perimeter groove (14a) intended to receive a tab (16b) of a fabric tightening member (16).

The transport containers (6) are left in georeferenced locations where the insects lay their eggs inside a rectangular trap (14). Once the eggs are placed in the rectangular trap (14), they are covered with a fabric (17a).

The fabric tightening member (16) is made up of a frame (16c) having a second central discharge (17) which inner face is covered with fabric (17a) which serves as barrier forcing the eggs and larvae or newborn specimens to fall into the rectangular trap (14). The outer (edge of the frame (16c) has a plurality of tabs (16a) connecting with the outer face of the rectangular trap (14). The fabric tightening member (16), together with the fabric (17a) are placed on the rectangular trap (14) once the eggs and larvae are placed in said trap.

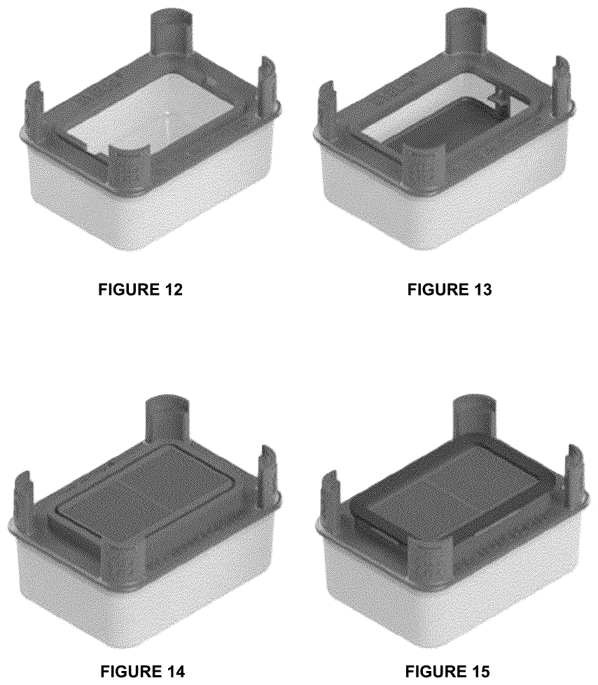

The organic waste placed on the inner floor (8) of the box (7) acts as a bait for the insects to lay on the fabric (17a) and drop thereon the eggs that are falling towards the plurality of slits (15) of the rectangular trap (14) and, afterwards, to the collecting tray (12). The assembly sequence of the transport container is shown in FIGS. 12 to 15.

Once the eggs and larvae have been collected, they are moved to a bioreactor having trays containing the organic waste that will be processed for them to be converted to an agricultural compost or fertilizer

### Bioreactor to Convert Organic Waste to High Value Added Products.

Secondly, the present invention also relates to a bioreactor (18) that converts organic waste to an agricultural compost or fertilizer.

The bioreactor (18) is comprised of a three-area module. Firstly, the reception area (19) for organic waste that is homogenized and texturized and later placed on trays (26).

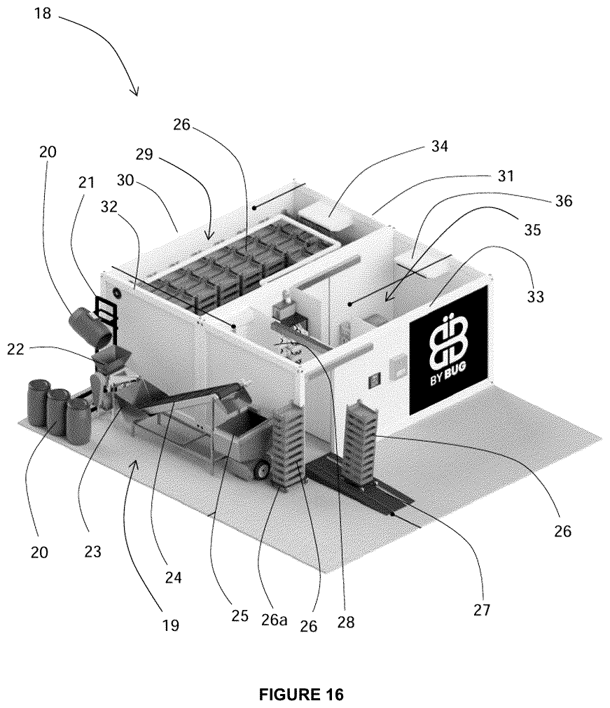

As may be observed in FIGS. 16 to 25, the reception area (19) starts to receive waste containers (20 (that are lifted up by a hoist car (21) and dropped into one or more grinding hoppers (22) wherein the waste is ground and homogenized to be, then, transferred to a conveying hopper (23) at which opening is an inclined conveying means (24) that may be an inclined conveyor belt or a screw conveyor, which lifts the waste and drops it to a collecting reservoir (25) wherein (it is given a foamy texture. The number of grinding hoppers is chosen based upon the hardness of the substrate with the harder substrate having more grinding hoppers. The grinding hoppers may be present in a sequential order or be present in parallel order.

The waste that is collected in the collecting reservoir (25) is transferred, through a hose, to a plurality of trays (26) that are stacked and loaded onto a car (26a) that conveys it through a ramp (27) towards a sanitation tunnel (28) where it is subjected to an ultraviolet (UV) bath. Subsequently, insect larvae are added for the organic waste treatment process to start.

Once the insect larvae have been added, the trays (26) are moved to a breeding area (29) being equipped with an air conditioning and humidifier system (34) to air condition the breeding area (29) based on the needs of the insect on the trays (26). The trays (26) are periodically inspected to verify if all of the organic waste has been converted to compost or fertilizer. Once this has taken place, the trays (26) are moved to a bio refining and packaging area (35) wherein the larvae are separated from the compost through a screen. After the aforementioned steps, the compost is packed and the larvae are dried in a microwave. Other possible devices that can be used to dehydrate the larvae include but are not limited to a dehydrator, an oven, a vacuum dryer, a freeze drier, a lyophilizer or other comparable equipment. In a variation, the device is a dehydrator. Likewise, the insect larvae are dried and ground to obtain a protein powder that will be sent to pellet manufacturers for animal feeding. The bio refining and packaging area (35) may have an air conditioning system (36) to keep temperature and humidity in the most suitable condition for the products that are being processed.

The sanitation tunnel (28), the breeding area (29), and the bio refining and packaging area (35) have walls forming an enclosure. These walls are: a front wall (33); a rear wall (30); a right wall (31) and a left wall (32).

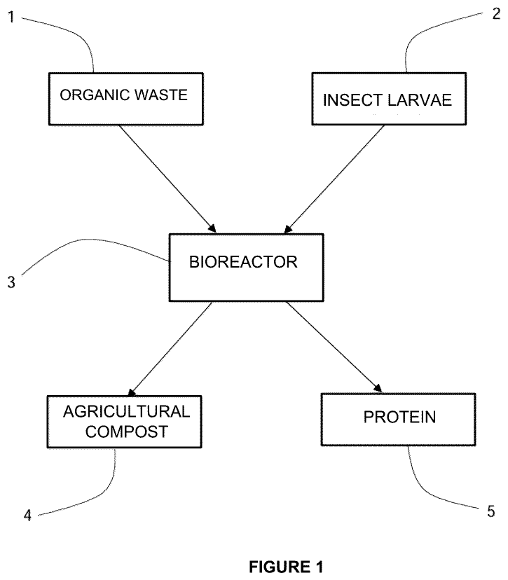

The following describes the figures of the present invention. FIG. 1 shows a block diagram with the input and output products in the bioreactor of the present invention. Organic waste 1 and insect larvae 2 are the input items and agricultural compost 4 and protein 5 derived from the insect larvae are the output items.

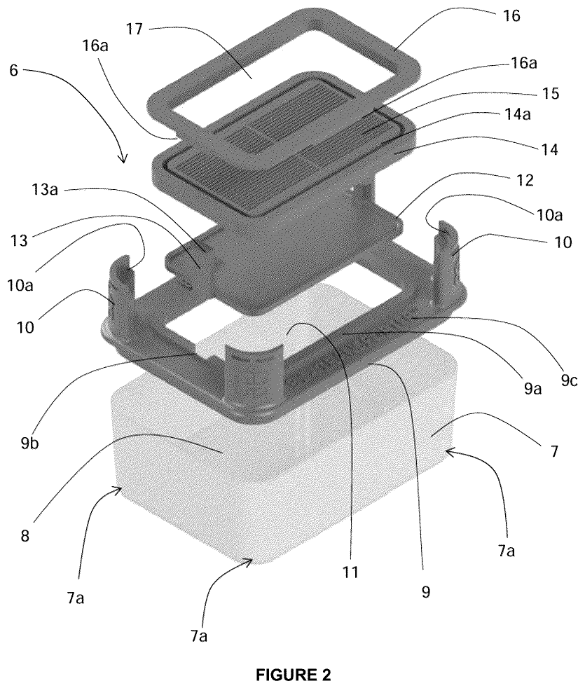

FIG. 2 is an exploded view of the transport container of the present invention. In an embodiment and as shown in this figure, there are five main parts to the transport container 6, a box 7, a lid 9, an egg and larva collecting tray 12, a rectangular trap 14, and a fabric tightening member 16.

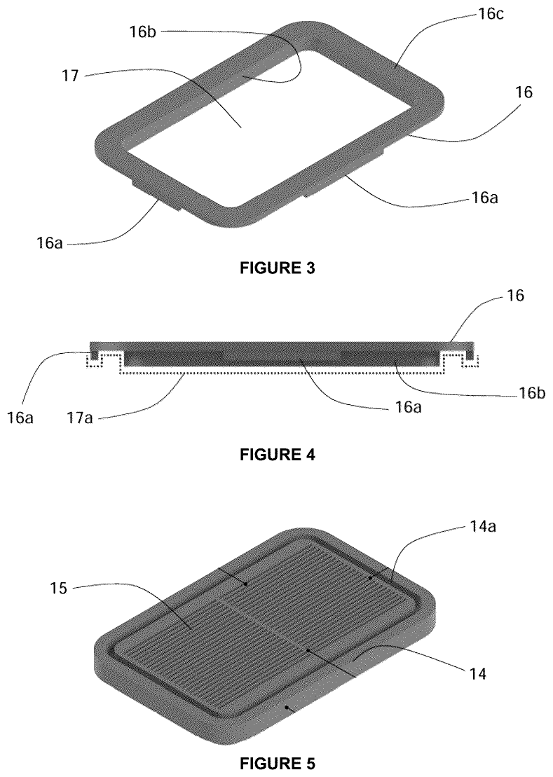

FIG. 3 shows a perspective view of the fabric tightening member used in the carrier container of the present invention.

FIG. 4 shows a side view of the fabric tightening member used in the carrier container of the present invention.

FIG. 5 is a perspective view of the rectangular (insect egg laying) trap 14 used in the transport container of the present invention.

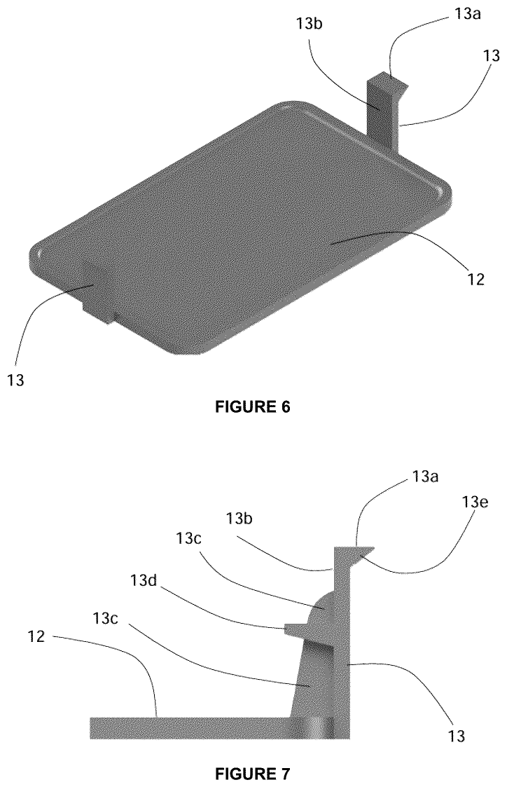

FIG. 6 shows a perspective view of the egg and larva collecting tray 12 used in the transport container of the present invention.

FIG. 7 shows a detailed side view of the rectangular pillar 13 of the collecting tray 12 used in the transport container 6 of the present invention.

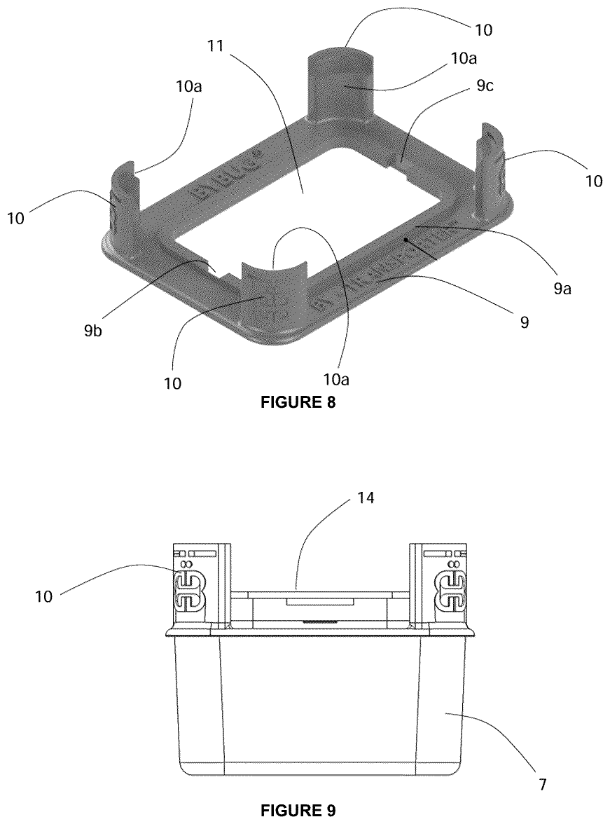

FIG. 8 shows is a perspective view of the lid 9 used in the transport container 6 of the present invention with the central discharge 11 shown. The concave cavity 10a on the vertical pillars 13 of the lid are apparent.

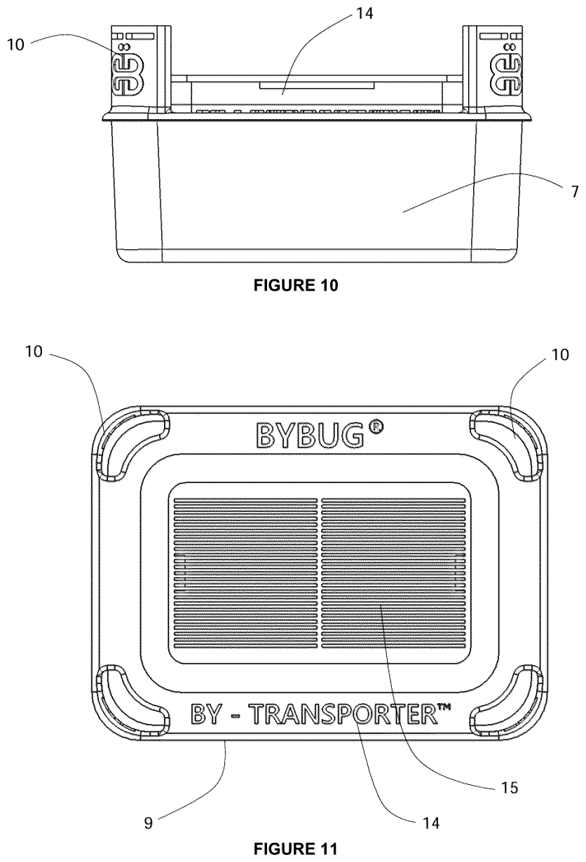

FIG. 9 is a front view of the transport container of the present invention with the five parts assembled. PLEASE LOOK AT FIG. 9. WHERE IS THE FABRIC TIGHTENING MEMBER 16? ALSO, THE RECTANGULAR TRAP 14 IN FIG. 9 DOES NOT APPEAR TO POINT TO THE SAME RECTANGULAR TRAP 14 IN FIG. 10.

FIG. 10 is a side view of the transport container of the present invention.

FIG. 11 is a top plan view of the transport container of the present invention showing the plurality of slits 15 on the rectangular trap 14.

FIGS. 12 to 15 show an assembly sequence of the transport container of the present invention.

FIG. 12 shows the transport container 6 with the box 7 and the lid 9.

FIG. 13 shows the transport container 6 with the box 7, the lid 9, and the egg and larva collecting tray 12. It should be noted that the egg and larva collecting tray is recessed in the box.

FIG. 14 shows the transport container 6 with the box 7, the lid 9, the egg and larva collecting tray 12, and the rectangular trap 14.

FIG. 15 shows the transport container 6 with the box 7, the lid 9, the egg and larva collecting tray 12, the rectangular trap 14, and the fabric tightening member 16.

FIG. 16 shows a perspective view of the bioreactor of the present invention with the three main areas being evident, a first area comprising a reception area 19, a second area comprising a breeding area 29, and a third area comprising a biorefining and packaging area 35.

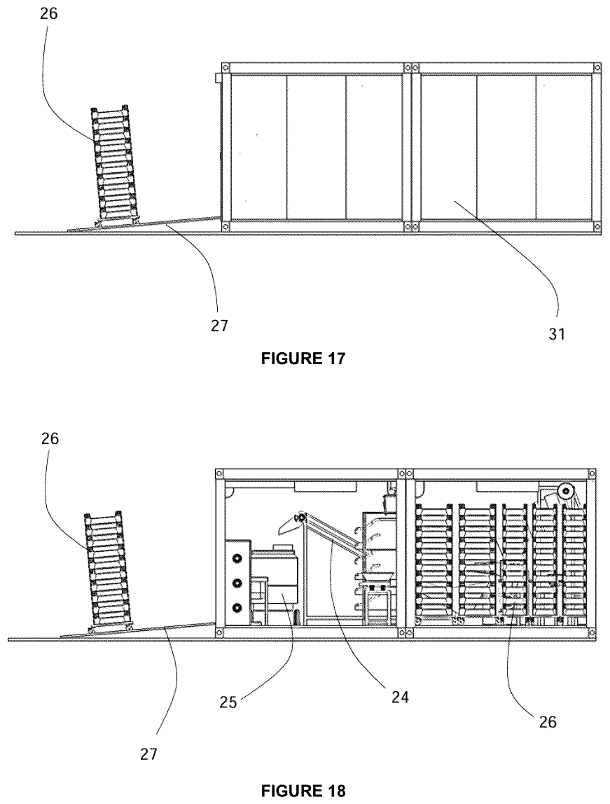

FIG. 17 is a right side view of the bioreactor of the present invention wherein one can see a plurality of trays 26 and a ramp 27 that is used to allow the trays to pass into the interior section of the bioreactor that is bordered on one side by right wall 31.

FIG. 18 is a right-side view without the perimeter walls of the bioreactor of the present invention. One again can see the plurality of trays 26 and a ramp 27 that is used to allow the plurality of trays 26 to pass into the interior section of the bioreactor. The collecting reservoir 25 and a conveying means 24 are also shown. In a variation, the conveying means is an inclined conveyor belt. It should be noted that the right-most plurality of trays 26 are shown in the breeding area 29 (see FIG. 16).

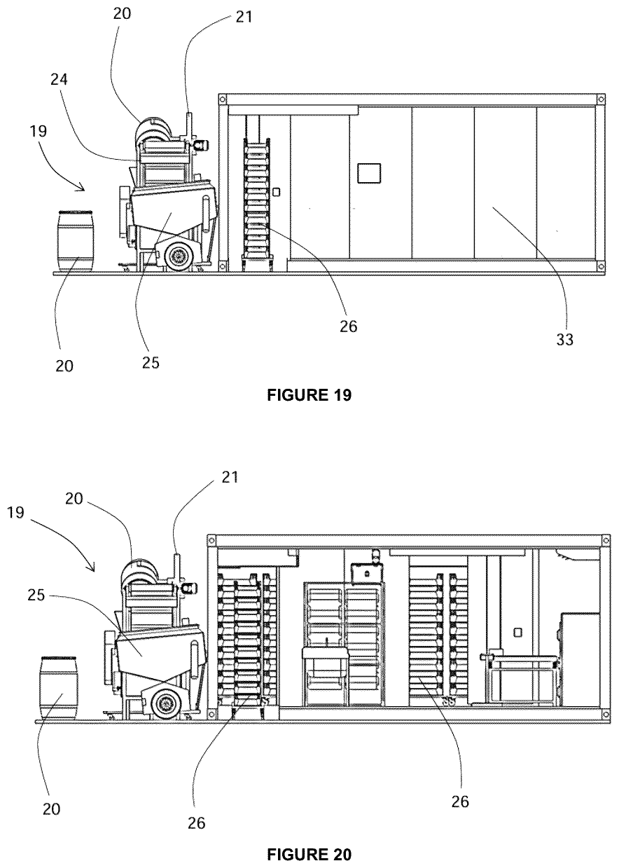

FIG. 19 is a front view of the bioreactor of the present invention. In this front view, the waste container 20 which is present in the reception area 19 can be seen. One waste container 20 can be seen which is ultimately feeding its contents into the collecting reservoir 25, via a hoist car 21 and a conveying means 24. The plurality of trays can also be seen in this figure along with front wall 33.

FIG. 20 is a front view without the perimeter walls (and in particular front wall 33) of the bioreactor of the present invention.

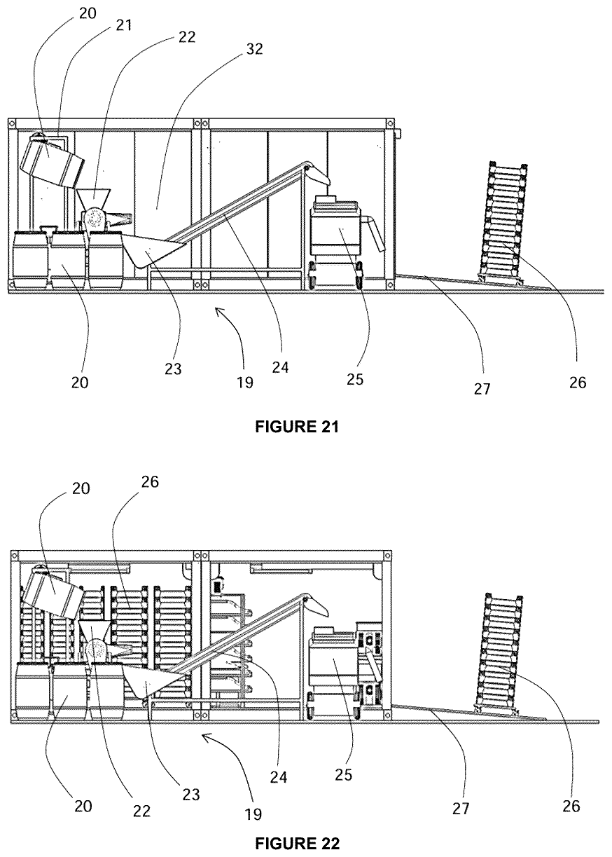

FIG. 21 is a left-side view of the bioreactor of the present invention. Waste containers 20 can be seen which is ultimately feeding its contents into the collecting reservoir 25, via a hoist car 21 and a conveying means 24. The plurality of trays 26 can also be seen in this figure along with left wall 32.

FIG. 22 is a left-side view without the perimeter walls (and in particular left wall 32) of the bioreactor of the present invention. Waste containers 20 can be seen which ultimately have their contents fed into the collecting reservoir 25, through the one or more grinding hoppers 22, the conveying hopper 23, and through conveying means 24. The plurality of trays 26 can also be seen in this figure and because left wall 32 is missing with the several plurality of trays 26 seen in the breeding area 29.

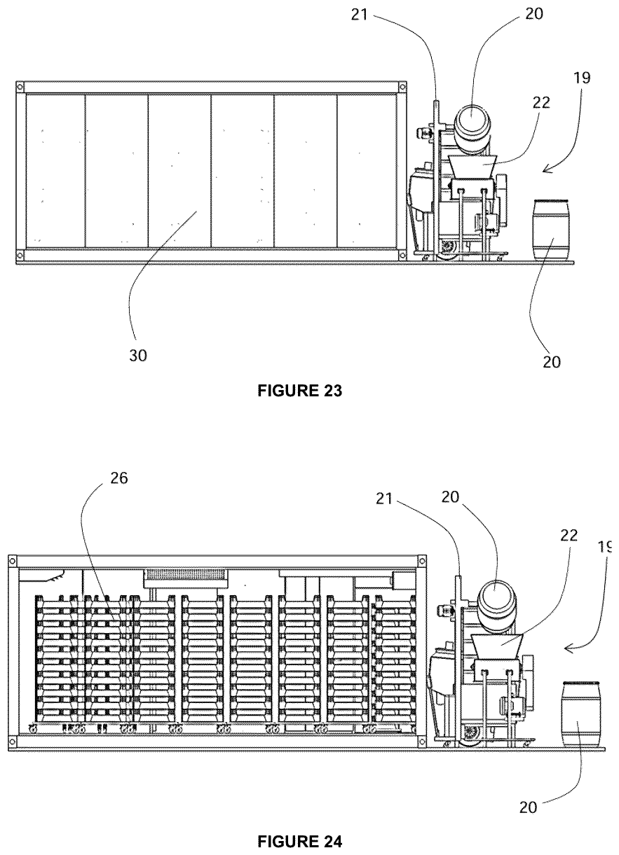

FIG. 23 is a rear view of the bioreactor of the present invention wherein one can see the back wall 30. Waste containers 20 can be seen which is ultimately feeding their contents into the collecting reservoir 25 (seen in FIG. 22), via a hoist car 21 and through the one or more grinding hoppers 22.

FIG. 24 is a rear view without the perimeter walls (and in particular rear wall 30) of the bioreactor of the present invention. Note that the components are the same as in FIG. 23, with a greater number of the plurality of trays 26 shown.

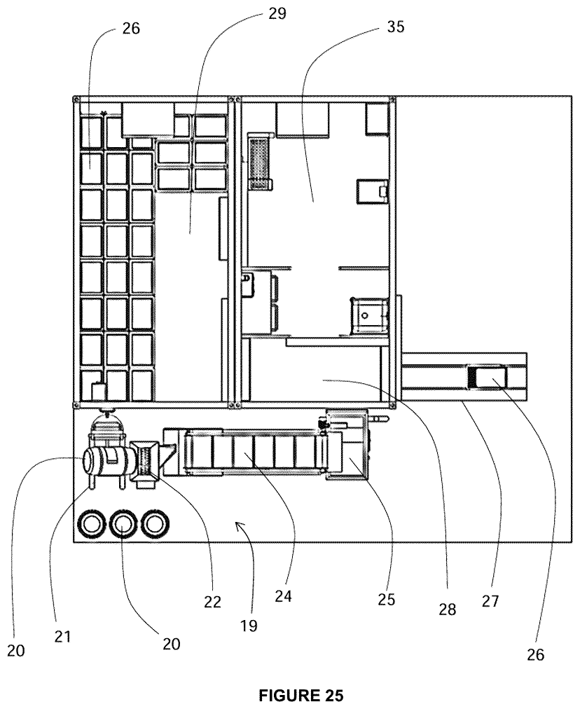

FIG. 25 is a top plan view of the bioreactor of the present invention. The first area comprising a reception area 19, a second area comprising a breeding area 29, and a third area comprising a biorefining and packaging area 35 are all evident. Sanitation tunnel 28 can also be seen. Waste containers 20 can be seen which is ultimately feeding their contents into the collecting reservoir 25 via a hoist car 21, through the one or more grinding hoppers 22, and through conveying means 24. The plurality of trays 26 can be seen going up ramp 27 and also in breeding area 29.

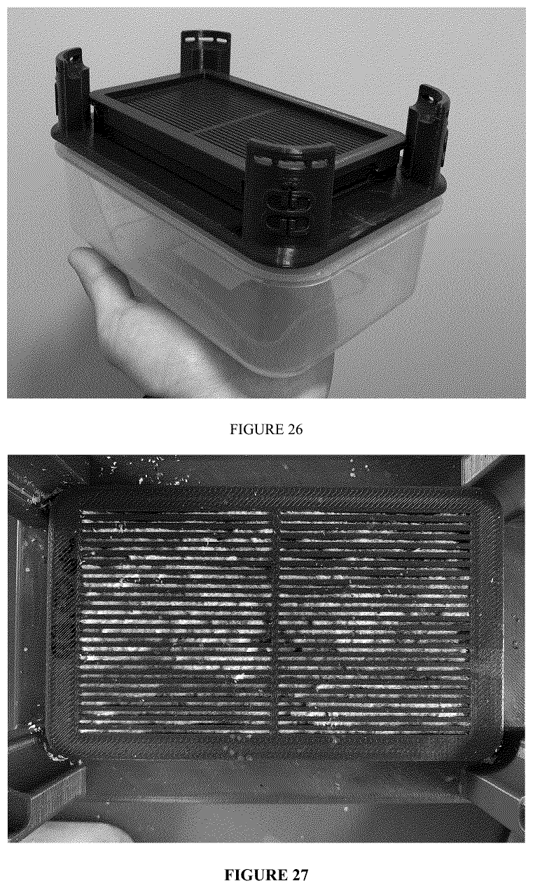

FIG. 26 shows a perspective view of the transport container 6 in its fully assembled state.

FIG. 27 shows a top down view of rectangular trap 14 with the plurality of slits that have eggs in them.

It should be understood based upon the disclosure above that the transport container is designed and configured to have the various components associated with it to be constructed and fit together in a manner that is compact, that serves its function, that allows it to be stacked, and that also allows it to be ideally suited to be used in a bioreactor for breeding of the insect larvae, and the processing of compost and/or fertilizer.

In an embodiment, the present invention relates to proteins obtained from insect larvae that are used to convert organic waste to animal feed and food for mammals, such as human beings. In an embodiment, the present invention also relates to an egg laying prompting reservoir, egg germinator and larva transporter, and a bioreactor that converts organic waste to high value-added goods using the insect larvae from the transporter.

In an embodiment, the present invention relates to systems for cultivating insects, systems for producing compost or fertilizer, systems for producing proteins associated with the cultivation of the insects, the associated products/devices/apparatuses necessary to cultivate insects and/or produce compost and/or fertilizer, including but not limited to the transport containers described herein, and the bioreactor described herein. Moreover, the present invention relates to methods/processes of achieving these aims. It should be understood that any feature that is described herein can be combined with any other feature as long as those features are not incompatible. For example, although the bioreactor has been described with three main areas, it should be understood that there may be fewer or more areas to the bioreactor than those described.

In an embodiment, the present invention relates to a system for insect cultivation, said system comprising a transport container (for insect larvae comprising a box having an inner floor for piling organic waste, the organic waste acting as an insect bait, the transport container comprising a lid made up of a lid frame which has a central discharge, the lid designed to fit on and sit atop the box, the lid frame having corners with vertical pillars emerging from the corners that have a concave cavity on an interior area to house a lower portion of the box to facilitate stacking of a second transport container, wherein a perimeter of the central discharge is contoured by a perimeter flange having at least one pair of rectangular recesses to accommodate an upper projection of at least one pair of rectangular pillars located at an outer corner of an egg and larva collecting tray, the rectangular recesses having an inclined inner face that operationally connects with an inclined lower face of said rectangular pillars, thus enabling the egg and larva collecting tray to hang from and below a plane of the lid.

In a variation, the transport container further comprises a rectangular trap and a fabric tightening member. In a variation, the rectangular trap is designed and configured to be operationally attached to the lid. In a variation, the fabric tightening member is designed and configured to be operationally attached to the rectangular trap. In a variation, the rectangular trap comprises a plurality of slits intended to catch eggs and larvae that fall into the egg and larva collecting tray. In a variation, an upper face of the rectangular trap has a perimeter groove designed and configured to receive a tab of the fabric tightening member. In a variation, the fabric tightening member comprises a frame having a second central discharge, said second central discharge designed and configured to be covered with a fabric, the fabric serving as a barrier forcing the eggs and larvae or newborn specimens to fall into the rectangular trap.

In an embodiment, the system further comprises a bioreactor. In a variation, the bioreactor comprises a three-area module, a first area, a second area, and a third area, the first area comprising a reception area, the second area comprising a breeding area, and the third area comprising a biorefining and packaging area. In a variation, the reception area comprises one or more grinding hoppers for grinding the organic waste, a conveying hopper, a collecting reservoir, a car, and a sanitation tunnel. In a variation, the car comprises ground waste that passes through the sanitation tunnel to the breeding area. In a variation, the sanitation tunnel comprises an ultraviolet (UV) bath. The UV bath irradiates the contents on the car (e.g. the ground waste) to kill bacteria or other pathogens. In a variation, the breeding area comprises at least one air conditioning and humidifying unit. In a variation, the breeding area is configured and designed to accept a plurality of egg and larva collecting tray trays, wherein the breeding area uses the at least one air conditioning and humidifying unit to provide ideal conditions for cultivating insects, and/or producing compost and/or fertilizer. In a variation, at least some of the plurality of egg and larva collecting trays are stacked. In a variation, the biorefining and packaging area comprises one or more of a second air conditioning and humidifying unit, and/or a screen. In a variation, the system further processes compost and/or fertilizer. In a variation, the biorefining and packaging area comprises the screen, and the screen is used to separate the larvae from the compost. In a variation, the system cultivates insects, and processes compost and fertilizer. IN a variation, the system is designed and configured to have a device that can dehydrate the larvae, said device comprising one or more of a microwave, a dehydrator, an oven, a vacuum dryer, a freeze drier, or a lyophilizer.

In an embodiment, the present invention relates to a method of cultivating insects, processing compost and/or fertilizer, wherein the method comprises using the system as described herein. In any event, the invention is claimed in the following claims.

We claim:

1. A system for insect cultivation, said system comprising a transport container (for insect larvae comprising a box having an inner floor for piling organic waste, the organic waste acting as an insect bait, the transport container comprising a lid made up of a lid frame which has a central discharge, the lid designed to fit on and sit atop the box, the lid frame having corners with vertical pillars emerging from the corners that have a concave cavity on an interior area to house a lower portion of the box to facilitate stacking of a second transport container, wherein a perimeter of the central discharge is contoured by a perimeter flange having at least one pair of rectangular recesses to accommodate an upper projection of at least one pair of rectangular pillars located at an outer corner of an egg and larva collecting tray, the rectangular recesses having an inclined inner face that operationally connects with an inclined lower face of said rectangular pillars, thus enabling the egg and larva collecting tray to hang from and below a plane of the lid.

2. The system of claim 1, wherein the transport container further comprises a rectangular trap and a fabric tightening member.

3. The system of claim 2, wherein the rectangular trap is designed and configured to be operationally attached to the lid.

4. The system of claim 3, wherein the fabric tightening member is designed and configured to be operationally attached to the rectangular trap.

5. The system of claim 4, wherein the rectangular trap comprises a plurality of slits intended to catch eggs and larvae that fall into the egg and larva collecting tray.

6. The system of claim 5, wherein an upper face of the rectangular trap has a perimeter groove designed and configured to receive a tab of the fabric tightening member.

7. The system of claim 6, wherein the fabric tightening member comprises a frame having a second central discharge, said second central discharge designed and configured to be covered with a fabric, the fabric serving as a barrier forcing the eggs and larvae or newborn specimens to fall into the rectangular trap.

8. The system of claim 7, further comprising a bioreactor.

9. The system of claim 8, wherein the bioreactor comprises a three-area module, a first area, a second area, and a third area, the first area comprising a reception area, the second area comprising a breeding area, and the third area comprising a biorefining and packaging area.

10. The system of claim 9, wherein the reception area comprises one or more grinding hoppers for grinding the organic waste, a conveying hopper, a collecting reservoir, a car, and a sanitation tunnel.

11. The system of claim 10, wherein the car comprises ground waste that passes through the sanitation tunnel to the breeding area.

12. The system of claim 11, wherein the sanitation tunnel comprises an ultraviolet (UV) bath.

13. The system of claim 12, wherein the breeding area comprises at least one air conditioning and humidifying unit.

14. The system of claim 13, wherein the breeding area is configured and designed to accept a plurality of egg and larva collecting tray trays, wherein the breeding area uses the at least one air conditioning and humidifying unit to provide ideal conditions for cultivating insects.

15. The system of claim 14, wherein at least some of the plurality of egg and larva collecting trays are stacked.

16. The system of claim 15, wherein the biorefining and packaging area comprises one or more of a second air conditioning and humidifying unit, and/or a screen.

17. The system of claim 16, wherein the system further processes compost and/or fertilizer.

18. The system of claim 17, wherein the biorefining and packaging area comprises the screen, and the screen is used to separate the larvae from the compost.

19. The system of claim 18, wherein the biorefining and packaging area comprises a means of dehydrating the larvae, the means being selected from the group consisting of a microwave, a dehydrator, an oven, a vacuum dryer, a freeze drier, and a lyophilizer.

20. A method of cultivating insects, processing compost and/or fertilizer, said method comprising using the system of claim 1.
